(19) United States
(12) Patent Application Publication
(10) Pub. No.: US 2025/0186376 A1
(43) Pub. Date: Jun. 12, 2025

(54) TOPICAL COMPOSITION FOR TREATING INJURIES OR CUTANEOUS INFLAMMATORY STATES OR PROCTOLOGICAL PATHOLOGIES

(71) Applicant: SILA S.P.A., Noale (IT)

(72) Inventor: Maurizio Lorenzon, Noale (IT)

(21) Appl. No.: 18/843,986

(22) PCT Filed: Mar. 8, 2023
(86) PCT No.: PCT/IB2023/052193

§ 371 Date: Sep. 4, 2024

(30) Foreign Application Priority Data
Mar. 8, 2022 (IT) 102022000004376

(51) Int. Cl. A61K 31/19; A61K 9/06; A61K 33/30; A61K 47/44; A61K 47/46

(52) U.S. Cl. CPC A61K 31/19; A61K 9/06; A61K 33/30; A61K 47/44; A61K 47/46

(57) ABSTRACT

There is described a topical composition for treating injuries or cutaneous inflammatory states or proctological pathologies, comprising butyric acid or a salt thereof at a fraction by weight between 1% and 50% and zinc oxide at a fraction by weight between 1% and 60%.

### TECHNICAL FIELD

The present invention relates to a novel topical composition for treating injuries or cutaneous inflammatory states or proctological pathologies, particularly indicated in the treatment of post-operative recovery.

### TECHNOLOGICAL BACKGROUND

In the treatment of cutaneous inflammatory states, such as, for example, dermatitis, eczema, skin rash, erythema, insect bites, or other types of inflammations, or generally in the treatment of cutaneous injuries, such as, for example, bedsores, it is known to use compositions for topical use, typically creams, based on steroid compounds, for example, cortisone compounds.

However, this family of active ingredients, while being effective in terms of the anti-inflammatory function thereof, may cause different collateral effects, including some serious ones, which discourage the use thereof or at least discourage the prolonged use thereof over time.

In the dermatological sector, it is further known to use topical compositions in the form of creams, pastes, gels or unguents, which contain hydrating and/or soothing compounds which promote the recovery of the correct functionality of the skin, or which contain “barrier” compounds, such as zinc oxide, which protects the skin from external agents which also have an antibacterial activity. A particular type of pathology, in which there are commonly used topical compositions for treating injuries or inflammatory states of the skin, is constituted by proctological pathologies or diseases which are linked to disturbances of the anal region, the anal canal and the rectum. Such pathologies are very common and the ones which are encountered most frequently are haemorrhoids, anal fissures, perianal fistulae, pilonidal cysts up to more severe conditions, such as prolapses, faecal incontinence and tumoral forms. Notwithstanding the fact that they are for the most part benign pathologies, they are highly debilitating diseases as a result of the painful states or other symptoms which negatively influence the quality of life of the patients who are suffering them. The cure may be pharmacological, both with topical treatments and via the systemic route, or may require surgical interventions.

One of the most frequent symptoms which is often associated with proctological pathologies is anal itchiness, a condition which is characterized by a powerful sensation of itchiness and/or burning which stimulates the patient who is suffering from it to continuously scratch until causing lesions and possible infections of the anal and perianal region. The treatment of anal itchiness, while depending on the cause which brings it about, provides in a first step for using symptomatic drugs, such as antihistamines or creams based on cortisone in order to alleviate the disturbance in order then to use a targeted therapy once the definitive diagnosis has been reached.

The cortisone-based creams are therefore widely used as a preferential treatment in order to reduce within short times the inflammatory effect and therefore the itchiness associated with the pathology. Many products for topical use for treating haemorrhoids also commonly contain some amount of corticosteroids which, over time, may bring about cutaneous atrophy, increasing the risk of bacterial or fungal supra-infections. As set out above, therefore, the prolonged use of topical preparations based on corticosteroids is discouraged.

In the same manner, for the treatment of post-operative recovery, it is normal practice to use creams or unguents based on pharmacological compounds which are capable of preventing any supra-infections, for example, antibiotic compounds, which may, however, develop over time types of pharmacological resistance, including at a systemic level, and the prolonged use of which may involve significant collateral effects.

The presence of the collateral effects indicated above further limits, on the one hand, the possibility of self-medication and, on the other hand, the possibility of controlling that the self-medication does not become the cause of additional problems for the patient. Another disadvantage of the topical compositions which are commonly used involves the fact that the excipient which is used is often an oil or grease which, particularly in the case of treatments in the anal or perianal region, may give rise to a sensation of discomfort in the patient.

In this field, therefore, there is felt the need to have available new compositions which allow an increasingly effective action of recovering the correct physiology of the skin and in particular of the skin in the anal and perianal region, and which do not as far as possible have collateral effects. In the more general sector of medical treatments, it is known that some compounds of n-butyric acid have advantageous biological effects on the digestive apparatus, in particular the trophic function thereof of the intestinal mucosa is known.

Butyric acid is a monocarboxylic fatty acid with a short chain (with 4 carbon atoms) which is classified among the volatile fatty acids (VFA) together with acetic acid (chain with 2 carbon atoms) and propionic acid (chain with 3 carbon atoms), which are generally known as SCFA (short-chain fatty acids). Butyric acid has two isomers, n-butyric acid and isobutyric acid. N-butyric acid melts at a temperature of approximately −8° C. and boils at approximately 164° C.

Among the compounds of n-butyric acid of greatest interest are the salts thereof, which are generally indicated as a “butyrate”, and particularly the sodium salt thereof.

The sodium salt of n-butyric acid is commercially available both in liquid form (in 50% aqueous solution as a direct product of the synthesis reaction of the sodium salt from n-butyric acid) and in the solid granular form (as a powder, white in colour, stable up to 250° C.).

The compounds of the n-butyric acid, in accordance with the surrounding environment, may be in dissociated form or in a non-dissociated form; the latter assumes a particular importance at a biological level because it can be absorbed by the intestinal walls and the cellular membranes of the microorganisms and can have a more pronounced effect with respect to the dissociated form.

The compounds of the n-butyric acid are mainly produced by carbohydrates (cellulose and starch) by fermentation under anaerobic conditions by different microorganisms and this process is also carried out in the large intestine. After the formation thereof, the butyrate is partially metabolized while the non-metabolized fraction of the non-dissociated butyrate is absorbed in the large intestine and enters the circulation.

As set out above, the compounds of the n-butyric acid have a trophic function of the intestinal mucosa with a resultant stimulation of the growth of intestinal villi.

The compounds of the n-butyric acid further influence the development of a number of gastro-enteric microorganisms.

However, the compounds of the n-butyric acid have a number of relevant disadvantages which limit the use and effectiveness thereof.

A first of these disadvantages is determined by the decidedly unpleasant odour of rancid butter which characterizes the initial n-butyric acid, which complicates the production and storage processes.

Furthermore, the compounds of the n-butyric acid are particularly sensitive to acid environments, where they can readily hydrolyse and reform the original n-butyric acid, as a result of which if they are administered per se, the n-butyric acid would immediately form at a gastric level, making it no longer available for the absorption thereof at the intestinal level.

In order to limit this disadvantage, it is known to micro-encapsulate the butyrate, covering it with a fatty matrix, as described, for example, in EP2352386, in the name of the same Applicant, which describes a production process for a compound based on n-butyric acid or the derivatives thereof which is micro-encapsulated in a fatty matrix based on saturated fatty acids and a mineral agent which allows protection of the derivatives of the n-butyric acid from the highly acidic environment which is present at the gastric level, but allowing the release thereof at the enteric level as a result of the degradation action of the fatty matrix by means of specific enzymes.

### DESCRIPTION OF THE INVENTION

The problem addressed by the present invention is to provide a novel topical composition for treating injuries or cutaneous inflammatory states or treating proctological pathologies which acts in the most rapid and effective manner possible without causing undesirable secondary effects.

This problem is solved by the present invention by using a topical composition according to the appended claims.

In particular, in a first aspect thereof, the topical composition of the present invention comprises a fraction by weight between 1% and 50% of n-butyric acid or a salt thereof and a fraction by weight between 1% and 60% of zinc oxide. The composition further comprises at least one pharmaceutically acceptable excipient.

The Applicant has observed that, as a result of the combined action of these two compounds, it is possible to obtain a composition which is capable of effectively and rapidly treating the dermatological and proctological pathologies and particularly of reducing the itchiness and substantially reducing the post-operative recovery times of injuries resulting from surgical interventions, especially in regions of the body which are extremely delicate, such as, for example, the anal and perianal region.

Furthermore, the Applicant has observed an effective anti-inflammatory and re-epithelializating activity of the topical composition of the invention, as a result of which it can be advantageously used in the treatment of injuries and inflammations of the skin, such as, for example, dermatitis, sun-burn, burns, erythema, itchiness, insect bites, abrasions and desquamations, irritations from radiotherapy and post-intervention sensitizations.

In particular, butyric acid or the salt thereof confers on the topical composition of the present invention high levels of anti-inflammatory, re-epithelializating, healing and trophic properties.

For its part, the zinc oxide confers on the topical composition of the present invention high levels of antibacterial, lenitive, protective and anti-inflammatory properties.

The Applicant has further verified that the composition of the present invention is also effective in the veterinary field for the treatment of injuries or inflammatory states of the skin of farm animals or domestic animals.

In the above-described aspect, the present invention may further have at least one of the preferred features described below.

In one embodiment, the butyric acid is in the form of n-butyric acid which at ambient temperature is in the liquid state. The n-butyric acid is in the substantially pure form with a fraction greater than 99%.

In an alternative embodiment, the butyric acid may be used in the form of sodium salt.

In one embodiment, the topical composition has a fraction by weight of the butyric acid or the salt thereof between 1% and 20%, preferably between 1% and 10%, more preferably between 2% and 7% and even more preferably of approximately 5%.

In one embodiment, the topical composition has a fraction by weight of zinc oxide between 1% and 30%, preferably between 1% and 15%, more preferably between 2% and 7%, and even more preferably of approximately 5%.

In one embodiment, the topical composition is in the form of creams or unguents or gels or pastes, in accordance with the specific desired applications. More preferably, the topical composition is in the form of a cream based on water.

This allows rapid absorption of the cream and this aspect is found to be particularly advantageous for use which is more comfortable by the patient both for cutaneous treatments and for treating proctological pathologies.

Preferably, the composition is a cream with a viscosity (measured at ambient temperature) between 4000 and 4500 centipoise and a pH between 7 and 7.5. In one embodiment, the topical composition comprises a fraction by weight of at least 40% of water, preferably a fraction by weight between 60% and 85%, more preferably approximately 75%.

In one embodiment, the topical composition comprises an emulsifying agent at a fraction by weight between 5% and 20%, preferably of approximately 10%.

Preferably, the emulsifying agent is selected from stearic acid, cetyl alcohol, stearyl alcohol or cetearyl alcohol or polyethylene glycol or the ethers thereof, polysorbate, silicons, docosahexaenoic acid (DHA), dimethylethanolamine (DMAE) or admixtures thereof.

In one embodiment, the topical composition comprises aloe vera at a fraction by weight from 0.1% to 5%, preferably of approximately 1%.

The provision of aloe vera at the percentages indicated above confers on the composition a better hydrating, healing and lenitive action.

In one embodiment, the topical composition comprises bisabolol at a fraction by weight from 0.05% to 1%, preferably at approximately 0.2%.

The provision of bisabolol at the percentages indicated above confers on the composition a better emollient, anti-reddening and lenitive action.

In one embodiment, the topical composition comprises:
a fraction by weight between 5% and 20%, preferably approximately 10%, of at least one emulsifying or stabilizing compound;
a fraction by weight between 2% and 7%, preferably approximately 5%, of sodium butyrate;
a fraction by weight between 2% and 7%, preferably approximately 5%, of zinc oxide;
a fraction by weight between 0.5% and 8%, preferably approximately 2.5%, of vegetable butter and/or vegetable oils;
a fraction by weight between 0.1% and 5%, preferably approximately 1%, of aloe vera;
a fraction by weight between 0.05% and 1%, preferably approximately 0.2%, of bisabolol;
a fraction by weight of water sufficient to reach 100%.

Optionally, the composition may comprise one or more from emollient compounds (from 1% to 10%), humectant compounds (from 0.1% to 1%), preservative compounds (from 0.1% to 2%), pH regulating compounds (from 0.1% to 2%) and odorizing compounds which are formed by natural and/or synthetic fragrances (from 0.1% to 2%).

Examples of emollient compounds suitable for the purposes of the invention are esters of fatty acids, fatty acids, polyols, vegetable butter oils and mineral oils. Preferably, the vegetable butter is shea butter and the vegetable oil is avocado oil and jojoba oil.

Examples of humectant compounds suitable for the purposes of the invention are glycols, polyalkylene glycols (such as polyethylene glycol, polypropylene glycol and butylene glycol) and the derivatives thereof, sorbitol, glycerol ethoxylate or propoxylate, xylitol.

Examples of preservative compounds suitable for the purposes of the invention are C1-C3 alkyl parabens, benzyl alcohol and the like, phenoxy ethanol.

Examples of pH regulating compounds suitable for the purposes of the invention are citric acid and malic acid.

Examples of odorizing compounds (natural and/or synthetic fragrances) suitable for the purposes of the invention are essential oils derived, for example, from lavender, cloves, camomile and citruses in general or pure compounds, such as, for example, eugenol, linalool and limonene.

In particular, eugenol and the essential oil of cloves, in addition to the above-mentioned aromatizing function, also have an antiseptic and anaesthetic activity, thereby supplementing the action of the zinc oxide.

In one embodiment, the topical composition is used for the treatment of injuries and inflammations of the skin, such as, for example, dermatitis, sun-burn, burns, erythema, itchiness, insect bites, abrasions and desquamations, irritations from radiotherapy and post-intervention sensitizations.

In one embodiment, the topical composition is used for the treatment of bedsores.

In one embodiment, the topical composition is used for the treatment of proctological pathologies, such as, for example, anal fissures, haemorrhoids, perianal fistulae or abscesses, pilonidal cysts or folds.

In one embodiment, the topical composition is used for the post-operative treatment of injuries resulting from surgical interventions in the anal or perianal region, such as, for example, interventions for removing haemorrhoids, fistulae, pilonidal cysts or folds, interventions for draining abscesses and resolving fissures.

The topical composition according to the present invention is preferably obtained by the following steps.

Beforehand, there is provision for melting waxes and oils (for example, shea butter and vegetable oils) at a temperature of approximately 75°, then there are successively added demineralized water, the pH regulating compounds, the humectants and the emulsifiers, and the whole is emulsified for a time necessary to obtain a homogeneous admixture.

Subsequently, there are added to the emulsion the zinc oxide and the butyric acid which are further mixed until obtaining a homogeneous and uniform admixture at the temperature of approximately 45°.

Finally, there are added the preservative agents, the aloe vera, the bisabolol and the odorizing compounds which are further mixed until obtaining a homogeneous admixture at the temperature of approximately 25°.

### EXAMPLE

With the above-described method, there has been produced a cream having the following composition (percentages by weight):
demineralized water: approximately 74.5%
emulsifying agents: approximately 10% (for example, aqueous solution of
cetearyl acid, cetrimonium chloride, ceteareth-20)
butyric acid: approximately 5%
zinc oxide: approximately 5%
aloe vera: approximately 1%
shea butter: approximately 1%
avocado oil: approximately 1%
jojoba oil: approximately 0.5%
preservative agents: approximately 0.6% (for example, benzyl alcohol, phenoxy ethanol)
humectant agents: approximately 0.4% (for example, xylitol)
aromatizing compounds (fragrances): approximately 0.55% (for example, eugenol, essential oils of lavender, cloves and camomile)
citric acid: approximately 0.25%
bisabolol: approximately 0.2%

The cream which is obtained in this manner has been tested in the treatment of proctological pathologies and in the post-operative recovery of surgical interventions in the anal and perianal region.

In particular, the cream has been tested on a sample of approximately 50 patients having different proctological pathologies, including: anal fissures, hemorrhoids, fistulae, pilonidal cysts, perianal dermatitis, anal itchiness, hypertrophic folds, a relevant portion of which were treated in the post-operative recovery following the surgical interventions (for example, removal) made necessary by the above-mentioned pathologies.

The cream was applied in the affected zone for two/three times per day and was well tolerated by all the patients without any significant undesirable effects.

In all cases, there was observed a substantial acceleration of the recovery processes with respect to the standard treatments, with a significant improvement of the oedematous component, the reddening and the mucosal congestion with an important reduction of the pain and the bleeding.

Furthermore, in the cases of post-operative treatment, there is noted an even more relevant reduction in the recovery times of the surgical injuries with a mean recovery time of approximately 15 days as compared with means of approximately 20 days typical of the standard treatments. In particular, there was noted a substantial improvement in the recovery from the inflammation and proliferation phases which are characterized by oedema, reddening, pain and the presence of granulation tissue, respectively. The surgical scars have also shown after the first days of application of the cream complete cleansing and a prompt achievement of the proliferative and remodelling recovery phases.

1. A topical composition for treating injuries or cutaneous inflammatory states or proctological pathologies, comprising:
butyric acid or a salt thereof at a fraction by weight between 1% and 50% and
zinc oxide at a fraction by weight between 1% and 60%.

2. The topical composition according to claim 1, wherein the fraction by weight of the butyric acid or the salt thereof is between 1% and 20%.

3. The topical composition according to claim 2, wherein the fraction by weight of the butyric acid or the salt thereof is between 1% and 10%.

4. The topical composition according to claim 3, wherein the fraction by weight of the butyric acid or the salt thereof is between 2% and 7%.

5. The topical composition according to claim 1, wherein the fraction by weight of the zinc oxide is between 1% and 30%.

6. The topical composition according to claim 5, wherein the fraction by weight of the zinc oxide is between 1% and 15%.

7. The topical composition according to claim 6, wherein the fraction by weight of the zinc oxide is between 2% and 7%.

8. The topical composition according to claim 1, comprising water at a fraction by weight greater than 40%.

9. The topical composition according to claim 8, wherein the fraction by weight of water is between 60% and 85%.

10. The topical composition according to claim 8, comprising at least one emulsifying agent at a fraction by weight between 5% and 20%.

11. The topical composition according to claim 1, comprising aloe vera at a fraction by weight between 0.1% and 5%.

12. The topical composition according to claim 1, comprising bisabolol at a fraction by weight between 0.05% and 1%.

13. The topical composition according to claim 1, comprising one or more compounds selected from eugenol, essential oil of lavender, essential oil of cloves and essential oil of chamomile, the one or more compounds being present in total at a fraction by weight between 0.1% and 2%.

14. The topical composition according to claim 1, for the post-operative treatment of injuries resulting from surgical interventions in the anal or perianal region or for treating anal itchiness.

15. The topical composition according to claim 1, for treating injuries or cutaneous inflammatory states in the veterinary field.

16. The topical composition according to claim 11, comprising aloe vera at a fraction by weight of approximately 1%.

17. The topical composition according to claim 12, comprising bisabolol at a fraction by weight of approximately 0.2%.

18. The topical composition according to claim 13, comprising one or more compounds selected from eugenol, essential oil of lavender, essential oil of cloves and essential oil of chamomile, the one or more compounds being present in total at a fraction by weight of approximately 0.55%.
